(19) United States
(12) Patent Application Publication
(10) Pub. No.: US 2006/0064050 A1
(43) Pub. Date: Mar. 23, 2006

(54) Wristlet cover for a CTS brace

(76) Inventor: Zana Wilkins Jackson, Fontana, CA (US)

Correspondence Address: STURM & FIX LLP, 206 SIXTH AVENUE, SUITE 1213, DES MOINES, IA, 50309-4076, US

(21) Appl. No.: 10/945,411

(22) Filed: Sep. 20, 2004

(51) Int. Cl. A61F 13/00; A61F 13/06

(52) U.S. Cl. 602/64; 602/60

(57) ABSTRACT

A wristlet cover device (10) for disguising the presence of a CTS brace (100) on a person's hand and wrist wherein, the cover device (10) includes a hollow sleeve member (20) made from a fabric material (21) having a decorative pattern (25) formed thereon wherein, the sleeve member (20) has distal (22) and proximal 23 openings and a thumb receiving opening (24) disposed proximate to, but spaced from the distal opening (22) and, wherein, at least the area adjacent said distal (22) and proximal (23) openings are elasticized.

### CROSS REFERENCE TO RELATED APPLICATIONS

Not applicable.

### BACKGROUND OF THE INVENTION

1. Field of the Invention

The present invention relates to the field of carpal tunnel syndrome braces in general and in particular for a wristlet cover that will disguise the presence of a brace.

2. Description of Related Art

As can be seen by reference to the following U.S. Pat. Nos. 4,986,265; Des. 323,217; 6,047,403; and, Des. 484,604, the prior art is replete with myriad and diverse decorative cast covers used to protect and keep clean the exterior of a cast.

While all of the aforementioned prior art constructions are more than adequate for the basic purpose and function for which they have been specifically designed, they are uniformly deficient with respect to their failure to provide a simple, efficient, and practical decorative cover specifically designed to cover and disguise the presence of a CTS brace in an aesthetically pleasing fashion.

As any woman who suffers from carpal tunnel syndrome is all too well aware, the conventional CTS braces have an unappealing utilitarian appearance that detracts from a woman's appearance particularly when they are dressed up for social functions and causes the attention of other people to be drawn to the brace rather than focusing on a woman's attire.

As a consequence of the foregoing situation, there has existed a longstanding need among female wearers of CTS braces for a new and improved wristlet cover device that would be sold in stylish pairs wherein the pair of wristlets would be seen as a fashion statement that is coordinated with their outfit when in reality one of the wristlets would actually comprise a decorative cover for a CTS brace; and, the provision of such an arrangement is the stated objective of the present invention.

### BRIEF SUMMARY OF THE INVENTION

Briefly stated, the wristlet cover for a CTS brace that forms the basis of the present invention comprises in general a slightly tubular sleeve member fabricated from a fabric material having a decorative pattern formed thereon wherein, the opposite ends of the sleeve member define openings for receiving a user's fingers and wrist and wherein, a thumb opening is provided on one side proximate to, but spaced from, the slightly wider end of the sleeve member.

As will be explained in greater detail further on in the specification, in one version of the preferred embodiment, the sleeve member may be formed from uniformly elasticized material such as spandex or the like that will provide a generally uniform, lightly gripping cover/enclosure over a CTS brace.

In addition, in a second version of the preferred embodiment, only the opposite ends of the sleeve member are elasticized which provides a much wider range of choices for the fabric material that forms the sleeve member such that the fabric material is complimentary to the fashionable attire that a woman is wearing.

It should further be noted that while this wristlet cover was specifically designed as a decorative cover for a CTS brace, it may also be worn in matched pairs either with or without the brace as a personal fashion statement which will substantially expand the useful life of this invention from a utilitarian standpoint.

### BRIEF DESCRIPTION OF THE SEVERAL VIEWS OF THE DRAWINGS

These and other attributes of the invention will become more clear upon a thorough study of the following description of the best mode for carrying out the invention, particularly when reviewed in conjunction with the drawings, wherein:

FIG. 1 is a perspective view of a shortened version of the first preferred embodiment of this invention;

FIG. 2 is a perspective view of an elongated version of the first preferred embodiment of this invention;

FIG. 3 is a perspective view of the second preferred embodiment of this invention; and,

FIG. 4 is a perspective view of a prior art CTS brace with the outer periphery of this invention depicted in phantom.

### DETAILED DESCRIPTION OF THE INVENTION

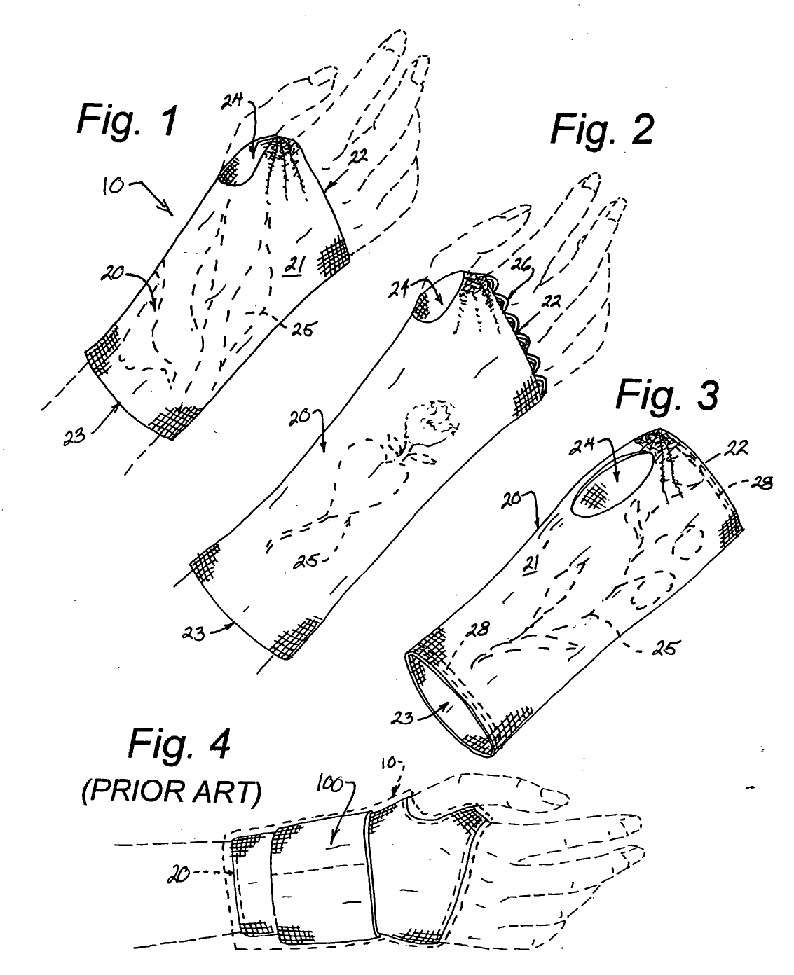

As can be seen by reference to the drawings, and in particular to FIG. 1, the wristlet cover device that forms basis of the present invention is designated generally by the reference number 10. The cover device 10 that forms the basis of the present invention comprises in general a tubular sleeve member 20 fabricated from a fabric material 21.

In addition, the tubular sleeve member 20 has an open distal end 22 dimensioned to accommodate a user's fingers and an open proximal end 23 dimensioned to accommodate the user's wrist wherein, a thumb receiving aperture 24 is also provided proximate to, but spaced from, the open distal end 22.

This basic structural arrangement holds true both for the first version of the preferred embodiment of the device 10 depicted in FIGS. 1 and 2, as well as, the second version of the preferred embodiment of the device 10′ depicted in FIG. 3.

Furthermore, both versions of the preferred embodiment have a decorative pattern 25 depicted in phantom formed on the outer surface of the fabric material 21 that constitutes the hollow sleeve member 20.

Turning now to FIGS. 1 and 2, it can seen that in the first version of the preferred embodiment of the cover device 10, the fabric material 21 is chosen from a class of fabrics that are generally uniformly resiliently expandable along one or more axis such as spandex or other elasticized material so as to provide a light, conformable fit of the device 10 in a closable fashion relative to a conventional CTS brace 100 as depicted in FIG. 5.

As can also be seen by reference to FIGS. 1 and 2, the tubular sleeve member 20 may be provided in a variety of different lengths that can either barely cover or substantially exceed the length of the CTS brace 100 depending on the personal preferences of the user.

In addition, the tubular sleeve member 20 may have a slightly tapered configuration wherein, the distal opening 22 is slightly larger than the proximal opening 23 due to the difference in circumference between the user's hand and their wrist.

Furthermore, additional decorative accessories 26 such as a lace trim 26 may be provided around the distal opening 22 to further disguise the utilitarian purpose of the device 10, as well as, to enhance the overall aesthetic appearance thereof.

As shown in FIG. 3, in the second version of the preferred embodiment of the device 10′, only the proximal 23 and distal 22 ends of the sleeve member 20 are elasticized by virtue of elasticized bands 28 (depicted in phantom) being incorporated into the proximal and distal cuff portions of the opposite ends of the sleeve member.

In addition, in this second version, the fabric material 21′ that constitutes the sleeve member 20 may be chosen from a wide variety of non-elastic material such as nylon, rayon, cotton, wool, velvet, etc., wherein, the particular fabric material 21′ employed is intended to be complementary in nature to the more popular fashion material selections that are popular during a particular time period.

Although only an exemplary embodiment of the invention has been described in detail above, those skilled in the art will readily appreciate that many modifications are possible without materially departing from the novel teachings and advantages of this invention. Accordingly, all such modifications are intended to be included within the scope of this invention as defined in the following claims.

Having thereby described the subject matter of the present invention, it should be apparent that many substitutions, modifications, and variations of the invention are possible in light of the above teachings. It is therefore to be understood that the invention as taught and described herein is only to be limited to the extent of the breadth and scope of the appended claims.

I claim:

1. A wristlet cover device for disguising the presence of a brace on a user's hand and wrist wherein, the cover device comprises
a hollow sleeve member fabricated from a fabric material having a decorative pattern associated therewith wherein, the sleeve member has a proximal opening, a distal opening, and a thumb receiving aperture disposed proximate to, but spaced from, said distal opening.

2. The cover device as in claim 1; wherein, said fabric material is chosen from a class of materials that are generally uniformly expandable along at least one axis.

3. The cover device as in claim 1; wherein, said fabric material is chosen from a class of materials that are generally inelastic but have cuff portions adjacent said proximal and distal openings that are elasticized.

4. The cover device as in claim 1 further including decorative accessories associated with the distal opening of the sleeve member.

5. The cover device as in claim 2 further including decorative accessories associated with the distal opening of the sleeve member.

6. The cover device as in claim 3 further including decorative accessories associated with the distal opening of the sleeve member.
